(19) United States
(12) Patent Application Publication
(10) Pub. No.: US 2022/0395271 A1
(43) Pub. Date: Dec. 15, 2022

(54) STAPLE CARTRIDGE WITH KNIFE SHARPENER

(71) Applicant: Covidien LP, Mansfield, MA (US)

(72) Inventors: Saumya Banerjee, Hamden, CT (US); Jacob C. Baril, Norwalk, CT (US); Justin Thomas, New Haven, CT (US); Garrett P. Ebersole, Hamden, CT (US); Matthew A. Dinino, Newington, CT (US); Roy J. Pilletere, Middletown, CT (US); Nicolette LaPierre Roy, Windsor Locks, CT (US)

(21) Appl. No.: 17/347,722

(22) Filed: Jun. 15, 2021

(51) Int. Cl. A61B 17/072; A61B 17/068

(52) U.S. Cl. CPC A61B 17/072; A61B 17/0686; A61B 2017/07285; A61B 2017/07271; A61B 2017/07264

(57) ABSTRACT

A surgical stapling device includes a replaceable staple cartridge that includes a knife sharpener for sharpening a cutting blade of the stapling device prior to each firing of the stapling device. In aspects of the disclosure, the staple cartridge includes wipers positioned distally of the knife sharpener to remove particulate material from the cutting blade before the cutting blade engages tissue.

### FIELD

This technology is generally related to surgical stapling devices, and more particularly, to surgical stapling devices including knife assemblies.

### BACKGROUND

Surgical stapling devices configured for endoscopic use are well known and commonly used during surgical procedures to minimize patient trauma and reduce patient recovery times. Typically, endoscopic stapling devices include a tool assembly and a drive assembly that is movable in relation to the tool assembly to actuate the tool assembly. The drive assembly includes a knife bar having a cutting blade for cutting tissue. The tool assembly includes anvil and cartridge assemblies that are coupled to each other by a pivot member and movable in relation to each other between open and clamped positions in response to movement of the drive assembly. The cartridge assembly includes a staple cartridge that supports staples that are ejected from the staple cartridge in response to movement of the drive assembly.

Some stapling devices include a staple cartridge that can be replaced after each firing of the stapling device to facilitate reuse of the stapling device. Other stapling devices include a reload assembly that includes a staple cartridge and a drive assembly that can be replaced after each firing of the stapling device to facilitate reuse of the stapling device. The use of a reload assembly provides a new cutting blade for each firing of the stapling device to maintain the quality of cutting. The use of a replaceable staple cartridge reduces the costs associated with a surgical procedure but generally requires reuse of the cutting blade. Repeated use of the same cutting blade may cause the cutting blade to become dull reducing the quality of cutting.

A continuing need exists for a surgical stapling device that can inexpensively provide a sharpened cutting blade through multiple firings of the stapling device.

### SUMMARY

One aspect of this disclosure is directed to a staple cartridge including a cartridge body, staples, and a knife sharpener. The cartridge body includes a proximal portion and a distal portion and defines a central knife slot and staple receiving pockets. The staple receiving pockets are positioned on opposite sides of the central knife slot and the staples are received within the staple receiving pockets. The knife sharpener is supported on the proximal portion of the cartridge body and is in axial alignment with the central knife slot.

Other aspects of the disclosure are directed to a surgical stapling device including an adapter assembly, a drive assembly, and a tool assembly. The adapter assembly has a proximal portion and a distal portion. The drive assembly includes a proximal portion and a distal portion and is movable between retracted and advanced positions. The distal portion of the drive assembly includes a cutting blade. The tool assembly includes an anvil and a cartridge assembly that are movable in relation to each other between open and clamped positions. The tool assembly receives the distal portion of the drive assembly. The cartridge assembly includes a channel member and a staple cartridge. The channel member defines a cavity, and the staple cartridge is received within the cavity of the channel member. The staple cartridge includes a cartridge body, staples, and a knife sharpener. The cartridge body has a proximal portion and a distal portion and defines a central knife slot and staple receiving pockets. The staple receiving pockets are positioned on opposite sides of the central knife slot. The staples are received within the staple receiving pockets of the cartridge body. The knife sharpener is supported on the proximal portion of the cartridge body and is in axial alignment with the central knife slot and with the cutting blade of the drive assembly.

In aspects of the disclosure, the cartridge body defines a cavity that is positioned proximally of and in communication with the central knife slot.

In some aspects of the disclosure, the cavity is defined by first and second opposed side walls of the cartridge body.

In certain aspects of the disclosure, the knife sharpener includes a first knife sharpener supported on the first side wall defining the cavity and a second knife sharpener supported on the second side wall defining the cavity.

In aspects of the disclosure, the first and second knife sharpeners extend from the first and second side walls, respectively, and are angled towards the central knife slot of the cartridge body in a distal direction.

In some aspects of the disclosure, the first and second knife sharpeners include abrasive surfaces.

In certain aspects of the disclosure, the first and second knife sharpeners are formed of a resilient material, and the abrasive surfaces include diamond edge sharpeners.

In aspects of the disclosure, the first and second knife sharpeners are supported in cantilevered fashion on the first and second side walls of the cartridge body.

In some aspects of the disclosure, the staple cartridge includes a wiper that is supported on the proximal portion of the cartridge body distally of the knife sharpener.

In certain aspects of the disclosure, the wiper includes a first wiper supported on the first side wall and a second wiper supported on the second side wall.

In aspects of the disclosure, the first and second wipers extend from the first and second side walls, respectively, and are angled towards the central knife slot of the cartridge body in a distal direction.

In some aspects of the disclosure, the first and second knife sharpeners are supported in cantilevered fashion on the first and second side walls of the cartridge body.

In certain aspects of the disclosure, the first and second wipers are formed from polyurethane.

Other features of the disclosure will be appreciated from the following description.

### BRIEF DESCRIPTION OF THE DRAWINGS

Various aspects of the disclosed surgical stapling device are described herein below with reference to the drawings, wherein:

FIG. 1 is a side perspective view of a surgical stapling device according to aspects of the disclosure with a tool assembly of the surgical stapling device in an open position;

FIG. 2 is side perspective view of a distal portion of the surgical stapling device shown in FIG. 1 with a staple cartridge separated from a channel member of a cartridge assembly of the surgical stapling device;

FIG. 3 is a side perspective view from above the staple cartridge shown in FIG. 2;

FIG. 4 is an enlarged view of the indicated area of detail shown in FIG. 3;

FIG. 5 is an exploded view of the proximal portion of the staple cartridge shown in FIG. 4;

FIG. 6 is a side perspective view of a drive assembly of the surgical stapling device shown in FIG. 1;

FIG. 7 is an enlarged view of the indicated area of detail shown in FIG. 6;

FIG. 8 is a side perspective view of the tool assembly of the stapling device shown in FIG. 1 with an anvil assembly of the tool assembly shown in phantom;

FIG. 9 is an enlarged view of the indicated area of detail shown in FIG. 8;

FIG. 10 is a cross-sectional view taken along section line 10-10 of FIG. 8;

FIG. 11 is an enlarged view of the indicated area of detail shown in FIG. 10 with the drive assembly in a retracted position;

FIG. 12 is an enlarged view of the indicated area of detail shown in FIG. 10 with the drive assembly advanced from the retracted position as a cutting blade of the drive assembly engages blade sharpeners;

FIG. 13 is an enlarged view of the indicated area of detail shown in FIG. 10 with the drive assembly advanced from the retracted position as a cutting blade of the drive assembly engages blade wipers of the cartridge assembly; and

FIG. 14 is an enlarged view of the indicated area of detail shown in FIG. 10 with the drive assembly advanced from the retracted position as a cutting blade of the drive assembly passes through the blade wipers.

### DETAILED DESCRIPTION

The disclosed surgical stapling device will now be described in detail with reference to the drawings in which like reference numerals designate identical or corresponding elements in each of the several views. However, it is to be understood that disclosed aspects of the surgical stapling device are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary fashion, while the term “distal” is used generally to refer to that portion of the device that is farther from the clinician during use of the device in its customary fashion. In addition, directional terms such as front, rear, upper, lower, top, bottom, and similar terms are used to assist in understanding the description and are not intended to limit the disclosure. Further, the term “clinician” is used generally to refer to medical personnel including doctors, nurses, surgeons, and support personnel.

The disclosure is directed to a surgical stapling device that includes a replaceable staple cartridge that includes a knife sharpener for sharpening a cutting blade of the stapling device prior to each firing of the stapling device. In aspects of the disclosure, the staple cartridge includes wipers positioned distally of the knife sharpener to remove particulate material from the cutting blade before the cutting blade engages tissue.

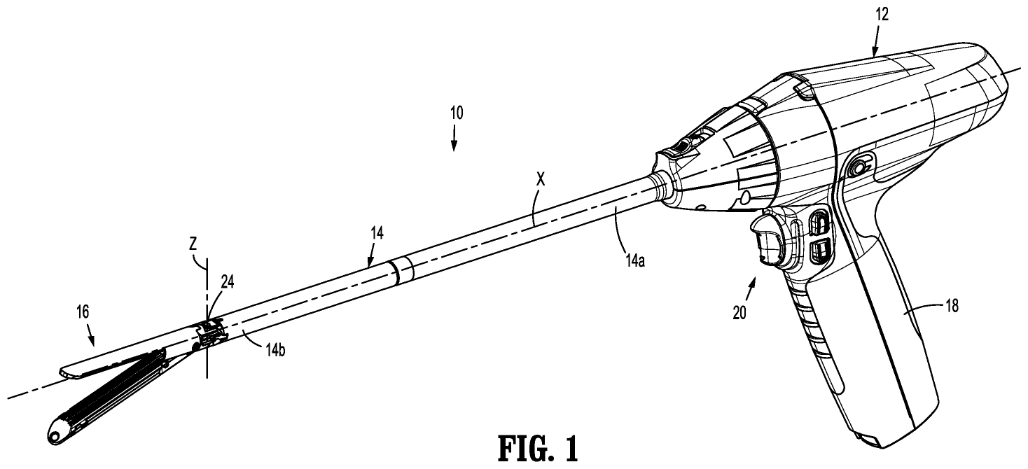

FIG. 1 illustrates a surgical stapling device shown generally as stapling device 10 that includes a handle assembly 12, an adapter assembly 14, and a tool assembly 16. The handle assembly 12 is powered and includes a stationary handgrip 18 and actuation buttons 20. The actuation buttons 20 are operable to actuate various functions of the tool assembly 16 via the adapter assembly 14, i.e., approximation of the tool assembly 16, and stapling and cutting of tissue. In certain aspects of the disclosure, the handle assembly 12 supports batteries (not shown) that provide power to the handle assembly 12 to operate the stapling device 10. Although the stapling device 10 is illustrated as a powered endoscopic stapling device, it is envisioned that the advantages of this disclosure are suitable for use with open-type stapling devices, manually powered surgical stapling devices, and robotically controlled stapling devices. U.S. Pat. No. 5,865,361 (“the '361 patent”) describes a stapling device that describes exemplary aspects of a manually powered stapling device.

The adapter assembly 14 defines a longitudinal axis “X” and includes a proximal portion 14a and a distal portion 14b. The proximal portion 14a of the adapter assembly 14 is coupled to the handle assembly 12. The distal portion 14b of the adapter assembly 14 is coupled to the tool assembly 16 by a pivot member 24 that defines an articulation axis “Z” that is transverse to the longitudinal axis “X” of the adapter assembly 14. The tool assembly 16 is pivotable about the articulation axis “Z” between a non-articulated position and articulated positions.

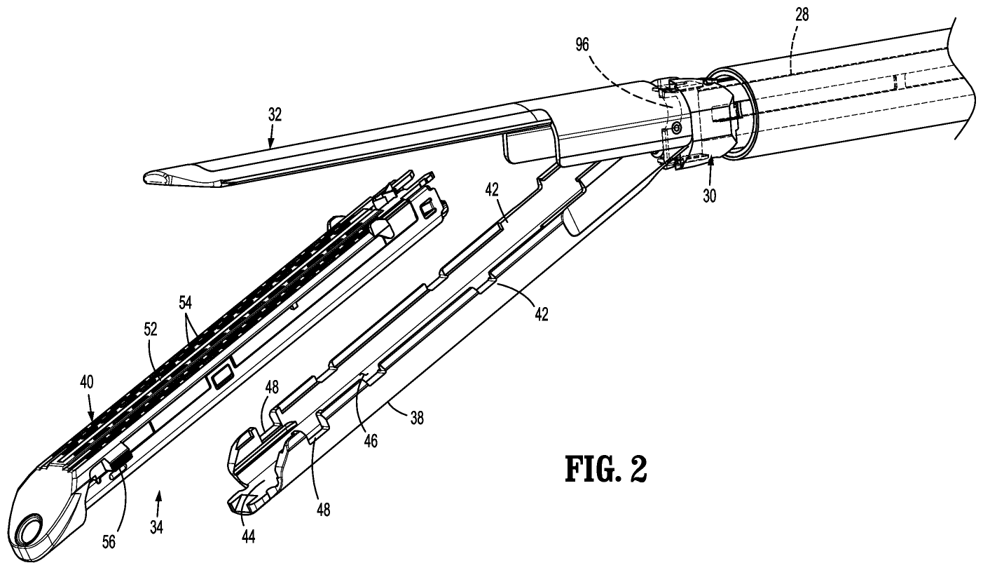

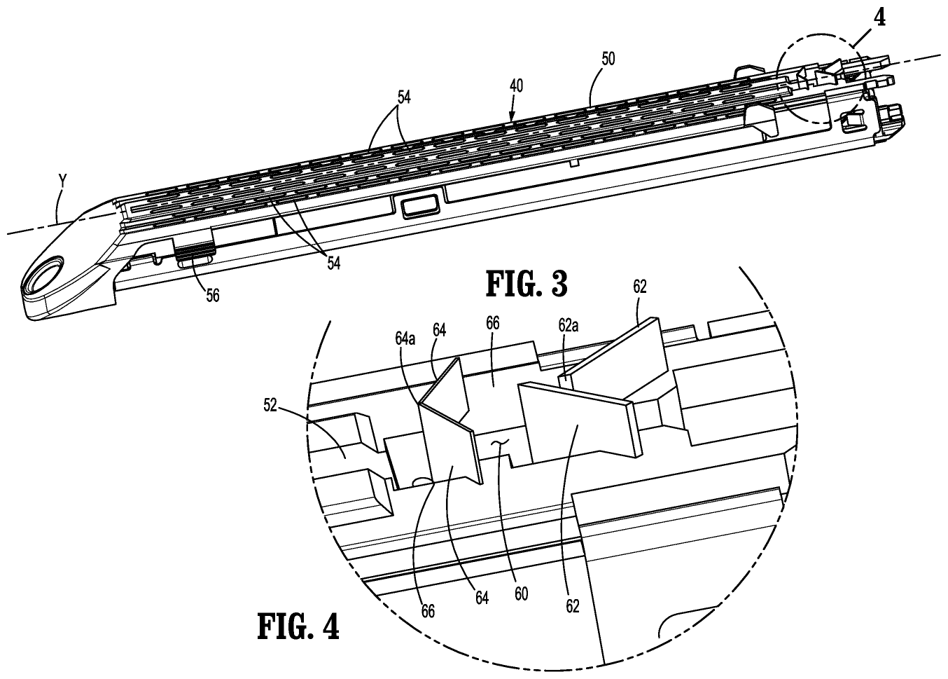

FIGS. 2 and 3 illustrate a distal portion of the stapling device 10 including the tool assembly 16 and the distal portion 14b of the adapter assembly 14. The distal portion 14b of the adapter assembly 14 includes a drive assembly 28 that extends from the adapter assembly 14 into the tool assembly 16 and is movable between retracted and advanced positions to actuate the tool assembly 16. The drive assembly 28 is described in further detail below.

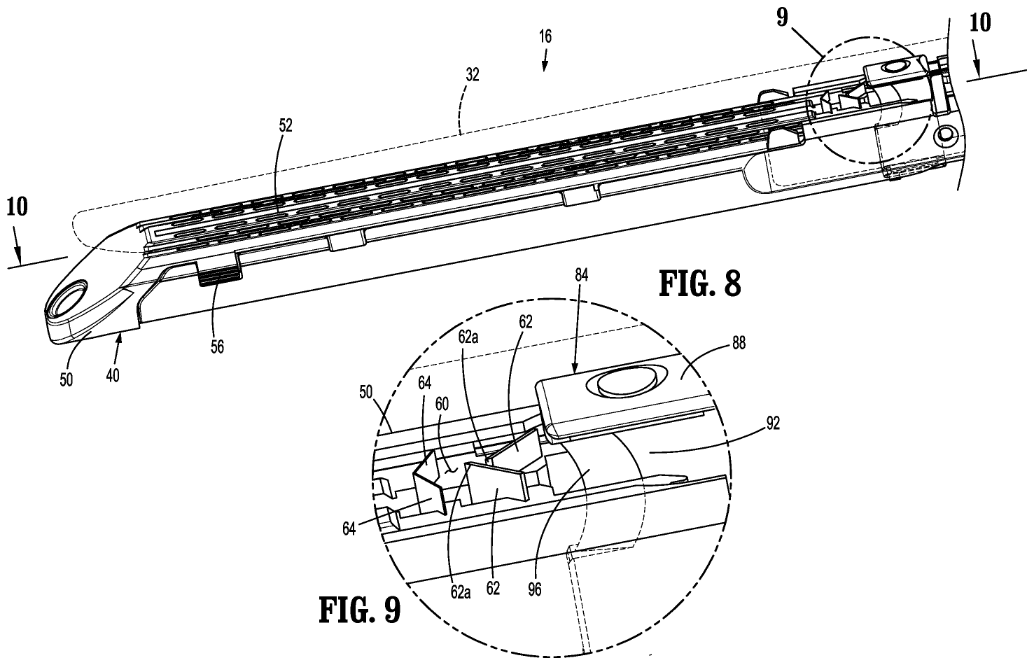

The tool assembly 16 includes a mounting assembly 30, an anvil 32, and a cartridge assembly 34. In aspects of the disclosure, the anvil 32 is fixedly secured to the mounting assembly 30 and the cartridge assembly 34 is pivotably secured to the mounting assembly 30 by pivot members 36 (only one is shown) such that the cartridge assembly 34 is movable in relation to the anvil 32 between an open position (FIG. 1) and a clamped position (FIG. 8). It is envisioned that the anvil 32 could be pivotably coupled to the mounting assembly 30 and the cartridge assembly 34 could be fixedly secured to the mounting assembly 30. In aspects of the disclosure, the pivot member 24 (FIG. 1) is formed with or coupled to the mounting assembly 30 to facilitate articulation of the tool assembly 16 about the articulation axis “Z” (FIG. 1).

The cartridge assembly 34 includes a channel member 38 and a staple cartridge 40. The channel member 38 is secured to the mounting assembly 30 and includes side walls 42 and a bottom wall 44 that define a cavity 46. The staple cartridge 40 is removably received within the cavity 46 of the channel member 38 and is replaceable to facilitate reuse of the stapling device 10. In aspects of the disclosure, the side walls 42 of the channel member 38 defines recesses 48.

FIGS. 3-5 illustrate the staple cartridge 40 which includes a cartridge body 50 that defines a central knife slot 52 and staple receiving pockets 54 positioned on each side of the central knife slot 52. The central knife slot 52 defines a longitudinal axis “Y” (FIG. 3) and extends through a proximal portion of the cartridge body 50. In aspects of the disclosure, the staple receiving pockets 54 are aligned in two or more rows on opposite sides of the central knife slot 52, e.g., three rows. The cartridge body 50 also includes laterally extending protrusions 56 (only one is shown) that are received within the recesses 48 (FIG. 2) of the channel member 38 to properly position the staple cartridge 40 within the cavity 46 of the channel member 38.

The proximal portion of the cartridge body 50 defines a cavity 60 that is positioned proximally of and communicates with the proximal end of the central knife slot 52. The cavity 60 is defined by side walls 66 that support a pair of knife sharpeners 62 and a pair of wipers 64. The knife sharpeners 62 are positioned proximally of the wipers 64 and include abrasive surfaces 62a that extend into the cavity 60. The abrasive surfaces 62a of the respective knife sharpeners 62 are aligned with the central knife slot 52 of the cartridge body 50 and are longitudinally offset from each other. Alternately, it is envisioned that the knife sharpeners 62 could be longitudinally aligned with each other. In aspects of the disclosure, the knife sharpeners 62 are formed of a resilient material, e.g., spring steel, and the abrasive surfaces 62a include diamond edge sharpeners. Alternately, other materials capable of performing the recited functions can be used to construct the knife sharpeners 62 and form the abrasive surfaces 62a. In aspects of the disclosure, the knife sharpeners 62 extend from the side walls 66 and are angled distally towards the axis “Y” defined by the central knife slot 52.

The wipers 64 extend from the side walls 66 defining the cavity 60 into the cavity 60 at a position distally of the knife sharpeners 62. The wipers 64 are angled in the distal direction towards the longitudinal axis “Y” defined by the central knife slot 52 and have distal ends 64a that are positioned adjacent each other and aligned with the central knife slot 52. In aspects of the disclosure, the wipers 64 are formed from a resilient material and function to remove particulate material from a cutting blade 70 (FIG. 7) of the drive assembly 28. In some aspects of the disclosure, the wipers 64 are formed from polyurethane although materials of construction are envisioned.

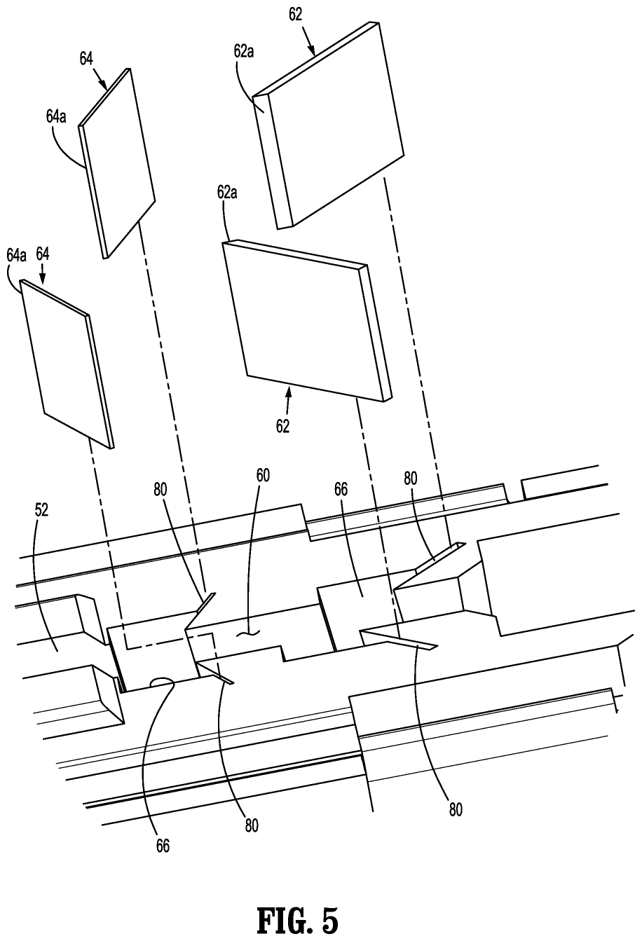

In aspects of the disclosure, the cartridge body 50 defines slots 80 (FIG. 5) that extend through the side walls 66 defining the cavity 60 in the cartridge body 50. The slots 80 are angled inwardly in the distal direction and receive a proximal portion of the knife sharpeners 62 and wipers 64 such that the knife sharpeners 62 and the wipers 64 are supported in cantilevered fashion on the cartridge body 50.

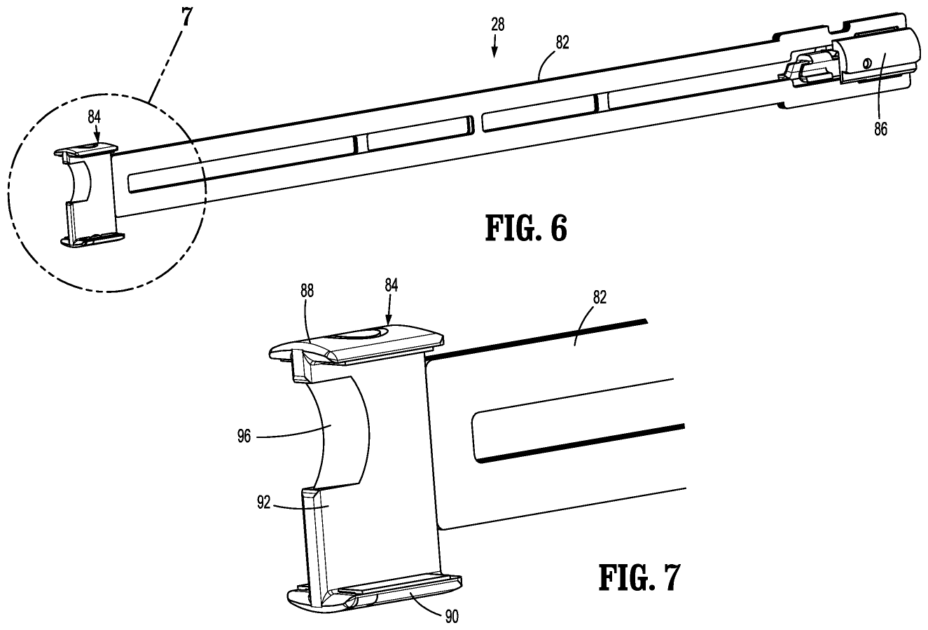

FIGS. 6 and 7 illustrate the drive assembly 28 which includes a resilient beam 82 and a working member 84. The resilient beam 82 which may be formed from a plurality of stacked or layered sheets has a proximal portion and a distal portion. The proximal portion supports a coupling member 86 that is adapted to couple the drive assembly 28 to a drive member (not shown) of the stapling device 10 (FIG. 1). The distal portion of the resilient beam 82 supports the working member 82. In aspects of the disclosure, the working member 82 is welded to the distal portion of the resilient beam 82 although other attachment techniques or materials are envisioned.

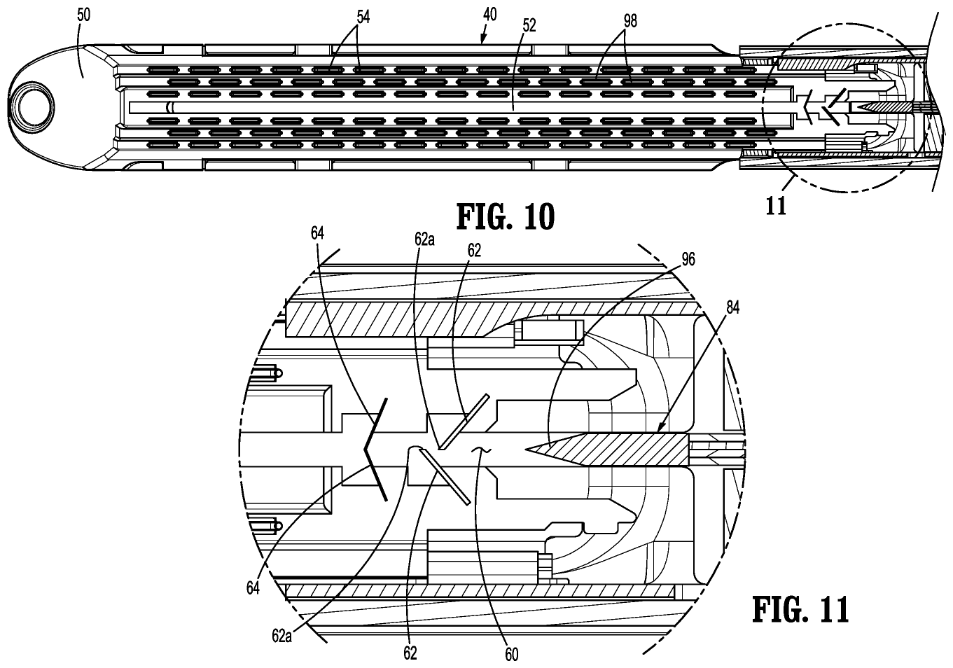

In aspects of the disclosure, the working member 84 has an I-beam configuration and includes a first beam 88, a second beam 90, and a vertical strut 92. The vertical strut 92 connects the first beam 88 to the second beam 90 and supports or is formed with a cutting blade 96. The drive assembly 28 is movable between a retracted position and an advanced position to actuate the tool assembly 16. More specifically, the drive assembly is movable from a retracted position to a clamped position to move the tool assembly 16 to the clamped position, and movable from the clamped position to an advanced position to eject staples 98 (FIG. 10) from the staple cartridge 40. For a more detailed description of the construction and operation of the drive assembly, see the '361 patent.

FIGS. 8-11 illustrate the drive assembly 28 of the stapling device 10 (FIG. 1) in the clamped position with the working member 84 of the drive assembly 28 supported within the proximal portion of the tool assembly 16. In the clamped position, the working member 84 of the drive assembly 28 is received in a proximal portion of the tool assembly 16 such that the cutting blade 96 of the working member 84 is positioned proximally of the cavity 60 of the staple cartridge 40 in alignment with the knife sharpeners 62 and the wipers 64.

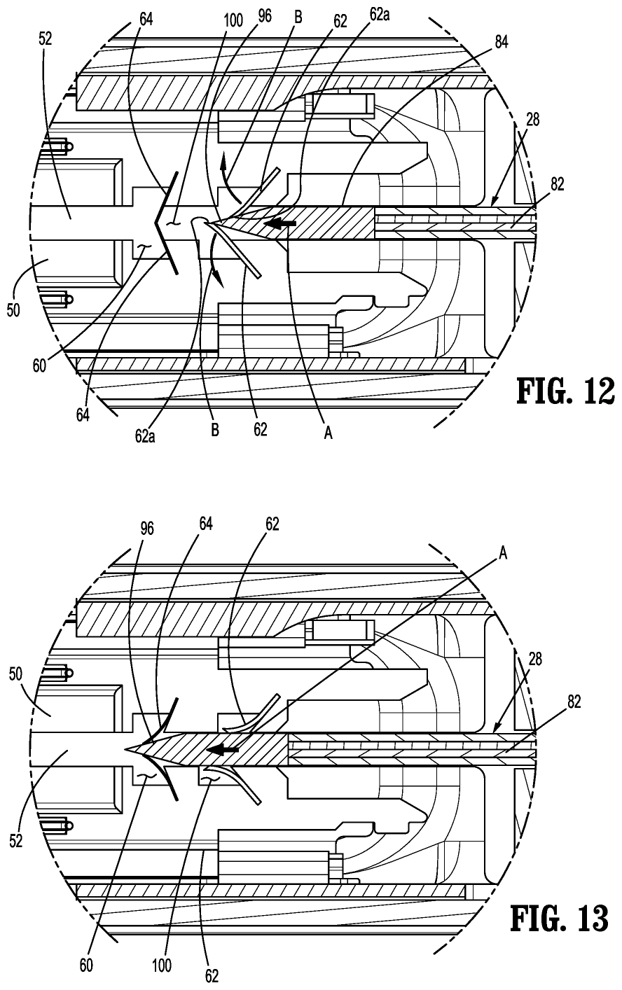

FIGS. 12-14 illustrate the proximal portion of the tool assembly 16 as the working member 84 of the drive assembly 28 moves through the proximal portion of the cartridge body 50. As the drive assembly 28 moves from its clamped position towards its advanced position in the direction of arrow “A” in FIG. 12, the cutting blade 96 engages and moves along the abrasive surfaces 62a of the knife sharpeners 62 to sharpen the cutting blade 96. As the cutting blade 96 engages the knife sharpeners 62, the knife sharpeners 62 are deformed outwardly in the direction of arrows “B” in FIG. 13 to allow the cutting blade 96 and the vertical strut 92 of the working member 84 to pass into the central knife slot 52 of the cartridge body 50. The outward deformation of the knife sharpeners 62 increases the force of the knife sharpeners 62 on the cutting blade 96.

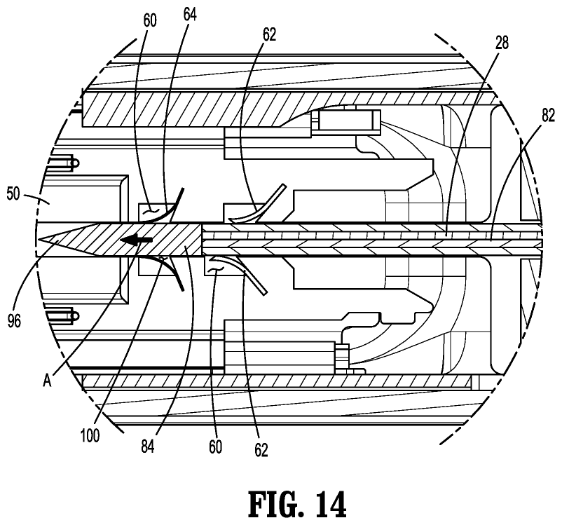

As the drive assembly 28 continues to move in the direction of arrows “A” in FIGS. 13 and 14, the cutting blade 96 moves past the knife sharpeners 62 and engages the wipers 64 which move along the outer surface of the cutting blade 96 as the drive assembly 28 is advanced within the cartridge body 50. The wipers 64 remove particulate material from the outer surface of the cutting blade 96 which may be present due to engagement between knife sharpeners 62 and the cutting blade 96. The particulate material is directed and collected in the trough portion 100 of the cavity 60 between the knife sharpeners 62 and the wipers 64.

After the stapling device 10 (FIG. 1) is fired, the drive assembly 28 is returned to its retracted position to move the tool assembly 16 to the open position. With the tool assembly 16 in the open position, the fired staple cartridge 40 is replaced with a fresh or unfired staple cartridge 40. It is noted that when the fresh staple cartridge 40 is inserted into the channel member 38 of the cartridge assembly 34, the working member 84 including the cutting blade 96 are positioned proximally of the knife sharpeners 62 and wipers 64.

In the disclosed stapling device 10 (FIG. 1), each time a new staple cartridge 40 is inserted into the cavity 46 defined by the channel member 38 (FIG. 2) and the stapling device 10 is fired, the cutting blade 96 is sharpened by the knife sharpeners 62. This allows the stapling device 10 to be fired multiple times using the same cutting blade 96 while minimizing any risk of tissue tearing caused by a dull cutting blade 96.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A staple cartridge comprising:
a cartridge body having a proximal portion and a distal portion and defining a central knife slot and staple receiving pockets, the staple receiving pockets positioned on opposite sides of the central knife slot;
staples received within the staple receiving pockets of the cartridge body; and
a knife sharpener supported on the proximal portion of the cartridge body, the knife sharpener in axial alignment with the central knife slot.

2. The staple cartridge of claim 1, wherein the cartridge body defines a cavity that is positioned proximally of and in communication with the central knife slot, the cavity defined by first and second opposed side walls of the cartridge body.

3. The staple cartridge of claim 2, wherein the knife sharpener includes a first knife sharpener supported on the first side wall defining the cavity and a second knife sharpener supported on the second side wall defining the cavity.

4. The staple cartridge of claim 3, wherein the first and second knife sharpeners extend from the first and second side walls, respectively, and are angled towards the central knife slot of the cartridge body in a distal direction.

5. The staple cartridge of claim 4, wherein the first and second knife sharpeners include abrasive surfaces.

6. The staple cartridge of claim 5, wherein the first and second knife sharpeners are formed of a resilient material, and the abrasive surfaces include diamond edge sharpeners.

7. The staple cartridge of claim 4, wherein the first and second knife sharpeners are supported in cantilevered fashion on the first and second side walls of the cartridge body.

8. The staple cartridge of claim 1, further including a wiper that is supported on the proximal portion of the cartridge body distally of the knife sharpener.

9. The staple cartridge of claim 8, wherein the cavity in the cartridge body is defined by first and second side walls, and the wiper includes a first wiper supported on the first side wall and a second wiper supported on the second side wall.

10. The staple cartridge of claim 9, wherein the first and second wipers extend from the first and second side walls, respectively, and are angled towards the central knife slot of the cartridge body in a distal direction.

11. The staple cartridge of claim 10, wherein the first and second knife sharpeners are supported in cantilevered fashion on the first and second side walls of the cartridge body.

12. The staple cartridge of claim 11, wherein the first and second wipers are formed from polyurethane.

13. A surgical stapling device comprising:
an adapter assembly having a proximal portion and a distal portion;
a drive assembly including a proximal portion and a distal portion and movable between retracted and advanced positions, the distal portion of the drive assembly including a cutting blade;
a tool assembly including an anvil and a cartridge assembly that are movable in relation to each other between open and clamped positions, the tool assembly receiving the distal portion of the drive assembly, the cartridge assembly including a channel member and a staple cartridge, the channel member defining a cavity and the staple cartridge received within the cavity of the channel member, the staple cartridge including:
a cartridge body having a proximal portion and a distal portion and defining a central knife slot and staple receiving pockets, the staple receiving pockets positioned on opposite sides of the central knife slot;
staples received within the staple receiving pockets of the cartridge body; and
a knife sharpener supported on the proximal portion of the cartridge body, the knife sharpener in axial alignment with the central knife slot and with the cutting blade of the drive assembly.

14. The stapling device of claim 13, wherein the cartridge body of the staple cartridge defines a cavity that is positioned proximally of and in communication with the central knife slot, the cavity defined by first and second opposed side walls of the cartridge body.

15. The stapling device of claim 14, wherein the knife sharpener of the staple cartridge includes a first knife sharpener supported on the first side wall defining the cavity and a second knife sharpener positioned on the second side wall defining the cavity, the first and second knife sharpeners supported in cantilevered fashion within the cavity of the cartridge body.

16. The stapling device of claim 15, wherein the first and second knife sharpeners extend from the first and second side walls defining the cavity in the cartridge body, respectively, and are angled towards the central knife slot of the cartridge body in a distal direction.

17. The stapling device of claim 16, wherein the first and second knife sharpeners are formed of a resilient material and have abrasive surfaces that include diamond edge sharpeners.

18. The stapling device of claim 13, wherein the staple cartridge further includes a wiper that is supported on the proximal portion of the cartridge body distally of the knife sharpener.

19. The stapling device of claim 18, wherein the cavity in the cartridge body is defined by first and second side walls, and the wiper includes a first wiper supported on the first side wall and a second wiper supported on the second side wall.

20. The stapling device of claim 19, wherein the first and second wipers extend from the first and second side walls, respectively, and are angled towards the central knife slot of the cartridge body in a distal direction.
